(19) United States
(12) Patent Application Publication
(10) Pub. No.: US 2004/0131704 A1
(43) Pub. Date: Jul. 8, 2004

(54) A method of organic and all natural treatment, prevention and relief of diaper dermatitis and other related skin irritations.

(75) Inventor: Tammy J. Morse, Wallingford, CT (US)

Correspondence Address: TAMMY J. MORSE, 44 KING STREET, WULLINGFORD, CT, 06492, US

(73) Assignee: Tammy J Morse, Wallingford, CT (US)

(21) Appl. No.: 10/248,274

(22) Filed: Jan. 3, 2003

(51) Int. Cl. A61K 7/06; A61K 35/78

(52) U.S. Cl. 424/725; 424/736; 424/764; 424/745

(57) ABSTRACT

A method of organic and all natural treatment, prevention and relief of diaper dermatitis and other related dermis irritations. The improved method is administered by topical application in the form of cream, ointment, salve, and spray, but not limited to, which contains, naturally derived anti-inflammatory, anti-microbial, anti-septic, anti-bacterial and anti-fungal agents.

### BACKGROUND OF INVENTION

Diaper dermatitis also known as diaper rash, is commonly caused by irritation in the diaper area. The rash is usually evident in the abdomen, genitalia and inside the skin folds of the thighs and buttocks and affects infants between the ages of 4 and 15 months. The severity can be mild to extreme, in some cases containing open sores or a secondary infection. Inflammation occurs as a result of prolonged exposure to irritants such as urine, stool and chemicals.

Over the years, diaper rash was thought to have been caused by numerous sources including teething, diet and ammonia in the urine. Medical experts now believe causes can include excessive amounts of moisture, rubbing and/or chaffing, or prolong contact of the skin with urine, or feces. Other possibilities include yeast or bacterial infections. There are some cases that have been linked to allergic reaction to chemicals in diapering and laundry products.

When skin stays wet for too long, the outer layers start to break down and is more easily damaged. Moisture from a soiled diaper can harm the skin making it more prone to chaffing.

Further rubbing between the moist folds of the skin only makes the rash worse. This is why diaper rash often forms in the skin folds of the groin and upper thighs.

More than half of babies between 4 months and 15 months of age develop diaper rash at least once in a two-month period. Diaper rash occurs more often as infants get older, mostly between 8 and 10 months of age.

Records of 272,841 encounters from the National Medical Care Survey (1990-1997) were examined for visits in which diaper dermatitis was diagnosed in children. There were approximately 8.2 million visits in which diaper dermatitis was diagnosed. For the pediatric population in the at-risk range, there was a 1 in 4 likelihood of being diagnosed with the skin disorder. Pediatricians provided 75% of services for the treatment of diaper dermatitis; the demographics of patients were similar to those of comparably aged individuals in the general population. [See Pediatrics & Adolescent Medicine, 2002;1 54:943-946]

According to U.S. Pat. No 6,419,963, Niazi, issued Jul. 16, 2002, it was only theory that the breakdown of the urine to yield ammonia primarily contributed to the formulation of diaper rash by increasing the alkalinity of the skin. It has been recently concluded that the prime factor to the cause of diaper rash is the feces or stool. In opposition to the alkaline pH due to bile. Recent studies have indicated that diaper rash is more prominent in the presence of feces than in the presence of urine, consequently providing a conceivable explanation for the problems with diaper rash relating to infants with frequent stools or experience diarrhea.

Infants taking antibiotics are more likely to get diaper rashes caused by yeast infections. Yeast infects the weakened skin and causes a bright red rash with red spots at its edges. Other data demonstrate that infants that are treated for otitis media (ear infections) with antibiotics (e.g.

Amoxicillin) were found to be at higher risk for diaper dermatitis compounded by a fungal infections such as Candida albicans (Honig et.al., 1988).

Currently the therapy for diaper rash is either a product with zinc oxide, Vitamins A and D or both. These active ingredients are combined with mineral oil, petrolatum, paraffin wax or lanolin.

The most popular products are available in the form of an ointment or water-in-oil emulsion. The thickness coupled with these products prevents the urine and feces from coming into direct contact with the skin by being repelled. The product acts as barrier inhibiting any diaper rash to form.

Desitin (Registered Trademark) Ointment, a product of Pfizer, Inc. is a very popular Over The Counter diaper dermatitis treatment, which uses a barrier method. The two common barrier substances contained in Desitin are zinc oxide and petrolatum. Additionally there are two common skin conditioning agents cod liver oil and lanolin. All of these agents are commonly used in topical skin conditioning preparations.

The U.S. Pat. No. 4,816,254 to Moss is an invention that provides an ointment composition for treating skin irritation such as diaper rash and decubitus. The composition includes zinc oxide, boric acid, karaya gum, Peruvian balsam, cod liver oil and an appropriate solvent and pharmaceutical carrier.

Another example of a diaper rash product is U.S. Pat. No. 4,556,560 issued to Buckingham.

This patent discloses and claims use of lipase inhibiting agents, such as the water soluble metallic salts including zinc chloride, in the treatment of diaper rash. The patent claims to treat diaper rash by inhibiting the deleterious effects of enzyme lipase action on the skin, said inhibition being achieved by incorporating and inhibitory agent of said lipase action into a barrier like carrier, said carrier having the characteristics of being relatively hydrophobic in nature thereby forming an effective barrier to the skin against urine and feces.

Yet another example of the prior art is U.S. Pat. No. 4,904,524 issued to Yoh. This patent disclosed and claims the encapsulation and micro bead formation and the use thereof the active silicone agent (Dimethicone) in the preparation of cloth wipes in the treatment of diaper rash.

U.S. Pat. No. 3,964,486, Blaney, issued Jun. 22, 1976, describes a disposable diaper or pad comprising an absorbent substrate having incorporated therein adipic acid in sufficient amount in inhibit ammonia formation and concomitant diaper rash. It is describes the use of adipic acid in the diaper at a level adequate enough to provide the urine with a pH in the range of 3.5 to about 5.5 during use throughout the entire diaper upon wetting with urine.

The U.S. Pat. No. 5,110,593 issued to Benford, May 5, 1992, is an invention that provides an ointment composition for the eradication and treatment of diaper dermatitis utilizing a skin conditioning agent, barrier agent and antimicrobial agent. The composition includes petrolatum, lanolin, and oxyquinoline.

Matravers claimed in U.S. Pat. No. 4,996,263 that a skin protective composition exhibiting enhanced water repellency and skin conditioning effects and contains aliphatic waxes and hydrophobic silicones. Specifically the use of an admixture consisting of a fatty acid admixture with one or more hydrophobic silicones.

Another example of a diaper rash product is U.S. Pat. No. 6,41 9,963 issued to Niazi, Jul. 16, 2002. In this composition the method for treatment of diaper rash uses natural products. The pharmaceutical composition contains beeswax, olive oil, and the herb Coptis chinesis Franch.

### SUMMARY OF INVENTION

In the present invention it is the primary objective to offer an organic and all natural alternative for the treatment, prevention and relief of diaper dermatitis and skin irritations for infants, children, and the elderly. The present invention comprises of Meadowfoam Seed Oil, Grapeseed Oil, Beeswax, and the herbs Chamomile, Comfrey, Calendula and Lavender.

### DETAILED DESCRIPTION

In general, the present organic and all natural composition for the treatment, prevention and relief of diaper dermatitis and other related skin irritations utilizes two oil mixtures. The first mixture is meadowfoam oil and the second being grapeseed oil. Other ingredients included are Beeswax, and the herbs, Chamomile, Calendula, Comfrey and Lavender.

In preparing the most preferred embodiment, the ointment is calculated on a 100 parts by percentage basis and includes 8-15% Beeswax, 8% - 10% meadowfoam oil, 60-80% grapeseed oil infused with 0.01-0.03% of the herbs Lavender, Comfrey, Chamomile, and Calendula. The grapeseed oil is infused with the herbs for a time period of but not limited to 48 hours. The two oil mixtures are heated between 143 and 151 F to achieve melt point of the beeswax. To the above mixture, beeswax is added and stirred continuosly until the desired formulation or 1 (one) hour. Thereafter, the product is cooled and inspected for quality control and then packaged.

In use, the composition may be applied to affected dermis where it will act as a barrier against further irritation, treat said area, proved relief and can be used as a prevention and method against further skin irritations.

The following research relating to ingredients in said invention have been chosen for the pharmacological properties outlined below.

Beeswax is. synthesized de novo by honey bees in four pair of glands located on the ventral side of the abdomen. Bees use the wax as their primary building material for making combs for rearing their blood and for the storage of honey and pollen. Beeswax is composed of a variety of monoester, diesters, hydroxylated esters, hydrocarbons and free fatty acids. This composition distinguishes the material as a wax rather than a fat. It is composed mostly of esters and long chained hydrocarbons, classic wax components. The major components of beeswax is palmitic acid esters, accounting for only 8% of beeswax composition. Triglycerides and diglycerides, typical of fats, are missing. The chemistry of beeswax components is ideal for the use of it by humans. These components, and the wax itself repeals water soluble materials but yet remains strong to temperatures of SO degrees C, and are reasonably flexible. They are not readily degraded or decomposed by moisture or microorganisms. Beeswax melts between 143 and 151 degrees F. Like most waxes, beeswax shrinks when cooled. The strength, flexibility and waterproofing qualities have made it excellent for addition to cosmetics and skin products. The gross chemical composition of beeswax below is illustrated. [See Schmidt J.O., Bee Products, Chemical Composition and Application. In: Bee Products Properties, Applications and Aipitherapy (Mizrahi, A. and Lensky, Y. Editors), p. 15-26. Plenum Press: New York. 1996]. [t1]

[Gross Chemical Composition of Beeswax]

| Chemical | Quantity % |
|---|---|
| Monoesters | 35% |
| Diesters | 14% |
| Triesters | 3% |
| Hydroxy Esters abd Polyesters | 12% |
| Acid Esters and Polyesters | 3% |
| Long Chained Hydrocarbons | 14% |
| Long Chained Fatty Acids | 12% |

Propolis is one example of healing property elements found in Beeswax. Much work has been conducted on the chemistry and properties of propolis. There have been hundreds of chemical compounds identified from propolis The main chemical classes present in propolis are flavonoids, phenolics, and various aromatic compounds. It is a bee product that is not clearly defined and varies from sample to sample. However, different propolis samples do share;considerable similarity in their physical and overall general chemical nature, thereby enabling a general discussion of the properties. Other properties include flavonoids which have excellent antioxidant, anti-bacterial, antifungal, anti-viral and anti-inflammatory properties. It is considered to be the one of the highest antimicrobial natural products. It acts against the widest spectrum of bacteria, fungi and viruses. In addition, it also contains anti-inflammatory, tumor cytotoxity and anesthetic effects.

Meadowfoam seed oil is different when compared to other vegetable oils. It contains over 98% fatty acids having chain lengths of 20 carbon atoms or more. Delta 5 monenoic and Delta 5, 13 dienoic acids are more stable to autoxidation than normal Delta 9, 12 acids found in other vegetable oils. An antioxidant is not usually necessary. The fatty acid composition typically is as follows: C20:1 is 63%, C22:1 is 16% and C22:2 is 17%. Meadowfoam seed oil is liquid at room temperature and one of the most stable liquids and is very resistant to oxidation. Due to the high stability of meadowfoam seed oil it is able to bond stability onto other systems. Unlike vegetable oils, meadowfoam seed oil contains 20-22 carbon chains. The typical vegetable oil contains only 16-18 carbon chains. It has the ability to hold up in the highest temperatures and is a better lubricant due to the longer chain which also provides for more stable fatty acids.

Grapeseed oil is rich in linoleic acid, an essential fatty acid very important for the skin and the cell membranes, it has allotted regenerative and reconstructing virtues which allow a better control of skin moisturization. In our invention we have chosen grapeseed oil for the emollient and film forming virtues. Grapeseed oil contains 0.8 to 1.5% unsaponifiables rich in phenols (topcopherols and steriods (campesterol, beta-sitosterol, stigmasterol). The chemical composition of grapeseed oil with average composition in fatty acids is as follows: Linoleic acid 58-78%, Oleic acid 12-28%, Palmitic acid 5-11% and Steric acid 3-6%.

According to the Department of Dermatology, Aberdeen Royal Infirmatory, Foresthill, Scotland, the following randomized trail of aromatherapy is successful treatment for alopecia areata. [see Hay IC, Jamieson M, Ormerod AD., Arch Dermatol 1998 Nov;1 34(11):1349-52. A randomized double blind, controlled trial of 7 month duration, with follow up at 3 and 7 months. Tests were conducted at Dermatology outpatient department. Eighty six patients diagnosed as having alopecia areata, divided into two groups. The active group massaged essential oils (thyme, rosemary, lavender, and cederwood) in a mixture of carrier oils (jojoba and grapeseed) into their scalps daily. The control groups used only carrier oils for their massage, also-daily. The outcome measures: Treatment success was evaluated on sequential photographs by 2 dermatologists (I.C.H.) and (A.D.O.) independently. The degree of improvement was measured by 2 methods, a 6-point scale and computerized analysis of traced areas of alopecia. Nineteen (44%) of 43 patients in the active group showed improvement compared with 6 (15%) of 41 patients in the control group (P=0.008). An alopecia scaled was applied in blinded observers on sequential photographs and was shown to be reproducible with good interobserver agreement (kappa=0.84). The degree of improvement on photographic assessment was significant (P=0.05). Demographic analysis showed that the 2 groups were well matched for prognostics factors. The conclusion, the results show aromatherapy to be a safe and effective treatment for alopecia areata. Treatment with these essential oils was significantly more effective than treatment with the carrier oil alone.

The following demonstrates how the cold acclimation or grapeseed oil feeding affects the phospholipid composition and mitochondria function in duckling skeletal muscle. [see Chainier F, Roussel D, Georges B, Meister R, RouanetjL, Duchamp C, Barre H., PubMed, Lipids 2000 October ;35 (1 0):1 099-106]. The phospholipid fatty acid (FA) composition and functional properties of skeletal muscle and liver mitochondria were examined in cold-acclimated (CA, 4 degrees C.) ducklings. Phospholipid FA of isolated muscle mitochondria from CA birds were longer and more unsaturated than those from thermoneutral (TN, 25 degrees C.) reared ducklings. The rise in long-chained and polyunsaturated FA (PUFA, mainly 20:4n-6) was associated with a higher State 4 respiration rate and a lower respiratory control ratio (RCR). Heptic mitochondria, by contract, were much less affected by cold acclimation. The cold-induced changes in phospholipid FA profile and functional properties of muscle mitochondria were reproduced by giving TN ducklings a diet enriched in grapeseed oil (GO, rich in n-6 FA), suggesting a casual relationship between the membrane structure and mitochondrial functional parameters. However, heptic mitochondria from ducklings fed the Go diet also showed an enrichment in long-chain PUFA but opposite changes in their biochemical characteristics (lower State 4, higher RCR). It is properties by membrane lipid compositions between skeletal muscle and liver may depend on muscle-specific factors possibly interacting with long-chain PUFA and affecting the proton leakiness of mitochondrial membranes.

Grapeseed oil as a safe and efficient hand cleansing agent. [see Krogsrud NE, Larsen Al., PubMed, Contact Dermatitis 1992 mar;26(3):208.

Chamomile is used on the skin for many different problems including poison ivy, chicken pox, diaper rash, and other kinds of rashes. In addition, it is used for eczema, hemorrhoids and cuts or scrapes. Studies have proven that chamomile may decrease irritation and swelling from rashes.

Important flavonoids have been identified in Chamomile including apigenin, luteotin, and quercetin. Recent research indicates that they display more or less inhibiting effects on certain malignant cell proliferation in vitro. [See, Agullo G,Gamet-Payrastre L, et al. Relationship between flavonoid structure and inhibition of phosphatidylinositol 3-kinase: a comparison with tyrosine kinase and protein kinase C inhabitation. Biochem Pharmacol].

Many different classes of active constituents have been isolated and used individually in medical practice and cosmetics. The plant contains 0.24%-1.9% volatile oil. The oil does not lose its potency and contains a-bisabolol (up to 50%) chamazulene cyclic sesquiterpenes, which directly reduces inflammation and are mild antibacterial. It also contains bisabolol oxides, farnesene and spiro-ether, which have anti-inflammatory and antispasmodic actions.

The anti-inflammatory effects of a hydroalcholic extract of chamomile recuita was tested in mice (1 ml. of extract contained 50 mg. of dry product). A 2.5 emulsion of croton oil was used on the ears of animals to produce edema. A dose dependant response was observed when chamomile extract was used to reduce edema. [See Tubaro A, Zilli C, et al. Evaluation of anti-inflammatory activity of a chamomile extract after topical application. 1 984;359].

The flowers of calendula og. calendula offinicalis contains sesquiterpene and flavonoid glycosides, triterpenoid saponins, sterols, fatty acids, carotenoids and other compounds. In vitro, calendula extracts displays uterotonic activity in isolated animal uteri. Data on antimicrobial effects are conflicting, but tend not to support use of calendula as an antimicrobial agent. In animals, calendula exerts sedative effects and synergistics effects with barbiturates in animal models. In two Polish studies from the 1960's, calendula exerted some estrogenic effects in ovariectomized mice. Calendula demonstrated moderate anti-inflammatory activity in several animal studies. Calendula extracts had anti-tumor effects in two studies in mice. In humans, Anecdotal reports and case series claim herbal mixtures including calendula can help heal gastric and duodenal ulcers and exert anti-inflammatory and wound healing effects. There are no controlled trials evaluating calendula use as a sedative, antimicrobial, estrogenic agent, uterine tonic, anti-tumor agent or vulnerary. [see Kemper M. D. M. P. H, Kathi J., Clinical Information Summary of Calendula, The Longwood Herbal Task Force, The Center for Holistic Pediatric Education and Research, 1999].

Herbs have been used in clinical medicine for thousands of years. However, it is only in recent times that we have been able to employ scientific methods to prove the efficacy of many of these herbs and to give us a better understanding of their mechanisms of action. This article will focus on the use of herbs in various dermatological conditions characterized by inflammation and pruritus. Topical preparations of many of these herbs are more commonplace in Europe. However, their availability is increasing in the US. As this is occurring we are witnessing a growing marriage between alternative and traditional medicines. [see Graf J., Skin Therapy Lett 2000;5(4):3-5].

By means of a bioassay-oriented fractionation of the CO2 extract of Calendula flowers, the triterpenoids are shown as the most important anti-inflammatory principles of the drug. Among them, the faradoil monoester appears to be the most relevant principle for the activity of the drug, due to its quantitative prevalence. The unesterfied faradiol, not present in the extract, is the most active of the tested compounds and equals indomethacian in activity, whereas the monools psi-taraxzsterol, lupeol, taraxasterol, and beta-amyrin are less active than the free diol. The anti-inflammatory activity of different CO2 extracts is proportional to their content of faradiol monoester, which can be taken as a suitable parameter for the quality control of Calendula preparations. [see Della Loggia R., Tubaro A, Sosa S., Becker H., Saar S., Issac O., Planta Med 1994 Dec;60(6):516-20].

The triterpene alcohols from calendula and other members of the daisy family have shown anti-inflammatory activity in the experimental mouse model. [see Akihisa T, Yasukawa K, Oinuma H, Kasahara Y, Yamanouchi S, Takido M, et al. Triterpene alcohols from the flowers of composite and their anti-inflammatory effects. Phytochemistry 1996; 43:1255-60].

One study reported enhanced vascularization in tissue cultures treated with a freeze-dried aqueous extract of calendula. [see Patrick K., Kumar S., Edwardson P, Hutchinson J. Induction of vascularization by an aqueous extract of the flowers of Calendula officinale L. The European marigold. Phytomedicine 1996; 3:11-18]. Among rats with surgical wounds, an ointment containing 5% of the flower extract of calendula plus allantoin significantly speeded healing. [see Kloucheck-Popova E., Popov A., Pavlova N., Krusteva S., Influence of the physiological regeneration and epithelialization using fractions isolated from Calendula officinalis. Acta Physiol Pharmacol BuIg 1982;8:63-7]. Other studies have shown that rats improved wound healing with 60% alcohol solution of calendula flowers. [see Rao S., Udupa A., Ydupa S., Rao P., Rao G., Kulkarni D. Calendula and hypericum: two homeopathic drugs promoting wound healing in rats. Fitoterapia 1991; 62:508]. There is a long tradition and numerous case reports if using calendula based ointments for wound healing and hemorrhoids. Among adults suffering from leprosy, an ointment containing 10% calendula extract appeared to help heal chronic skin sores and prevent-additional infections. [see Kartikeyan S., Chaturvedi R. M., Narkar S. V. Effect of calendula on topic ulcers. Lepr Zrev 1990; 61:399].

Comfrey contains an abundance of allantoin. Allantoin in aqueous solution in strengths of 0.30 percent has a powerful action in strengthening epithelial formations, and is a valuable remedy not only in external ulceration, but also in ulcers of the stomach and duodenum. Comfrey Root is used as a source of cell proliferant Allantoin, employed in the dealing of chronic wounds, burns, ulcers, etc. [see Macalister, British Medical Journal, Jan. 6, 1912].

Plants used in Swedish traditional medicine to treat inflammatory diseases and/or wounds were selected, based on literature data, for evaluation of inhibitory activity on prostaglandin biosynthesis and platelet activating factor (PAF) induced exocytosis in vitro. Fifty-nine water extracts from 52 different plants in 28 families were tested. A number of plants, e.g. Calluna vulgaris, Corylus avellana, Geum urbanum, Juniperus communis, Polygonum aviculare, Potentilla erecta and Salix caprea were found to be active in both assays. The most potent cyclooxygenase inhibitors were extracts of Calluna vulgaris, Potentilla erecta, and Salix caprea. None of the extracts inhibited just the prostaglandin biosysnthesis. In the PAF-test, high inhibition was obtained by 19 extracts, the most potent of which were from Geum rivale, G. urbanum, Solanum dulcamara, Symphytum x uplandicum and Vaccinium vitis-idaea. The in vitro effects in relation to the traditional use, chemical contects and botanical classification, as well as the possibilities and the limitations of the methods are discussed. [see Tunon H., Olavsdotter c, Bohlin L., Evualation of anti-inflammatory activity of some Swedish medicinal plants. Inhibition of prostaglandin biosynthesis and PAF-induced exocytosis. J Ethnopharmacol 1995 Oct;48(2):61-76].

The therapeutic properties of Lavender include: Antiseptic, analgesic, anti-convulsant, anti-depressant, anti-rheumatic, anti-toxic, anti-spasmodic, anti-inflammatory, emmenagogue, anti-toxic, carminative, deodorant, diuretic, nervine, restorative, sedative, insecticide and tonic. Lavender oil has in vitro antimicrobial activity against bacteria, fungi, and some insects. The essential oil exerts spasmolytic activity in smooth muscle in vivo, supporting its historical use as a digestive aid. Over 150 compounds have been isolated from the oil. Although the chemical composition of these oils are complex, the biological activities of the major chemical species present have been evaluated.

Linalyl acetate and linalool sedative and local analeptic effects as well as antibacterials, antifungal and insecticidal effects. [see Ghelardini C, Galeotti N., Slavatore G., Mazzanti G., Local anaesthetic activity of the essential oil of Lavandula augustifolia. Planta Med 1999; 65:700-3]. Following topical application of Lavender oil, linalyl acetate and linool can be detected in the blood within five minutes, peak at 19 minutes and are cleared within 90 minutes. [see Jager W, Buchbauer G, Jirovetz L, Fritzer M. Percutaneous absorption of lavender oil from a massage oil. Journal of the Society of Cosmetic Chemists 1992; 43:49-54]. Among children hospitalized for HIV, massage with a combination of several essential oils including L. angustifolia appeared to decrease the need for analgesic medication and entirely relieve the persistent pain of some children. [see Styles J. The use of armotherapy in hospitalized children with HIV. Complement ther Nurs 1997; 3:16-20]. In a randomized controlled clinical trial among 100 patients in a critical care unit, received lavender armotherapy combined with massage resulted on a 50% reduction in reported pain and a reduction in heart rate in 90% of participants. [see Wolfson A, Hewitt D. Intensive armocare. IntJ Aroma 1992; 4:12-14].

Differnet lavender species have variable antibacterial effects, depending on the concentration of specific chemical constituents. The essential oil of L. angustifolia has bacteriostatic and bactericidal activity against methicillin-resistant staphylococcus aureas and vancomycin-resistant Enterococcus aecium. [see Nelson RRS. In vitro activities of five plant essential oils against methicillin-restiant Staphylococcus aureaus and vancomycin-restiant Enterococcus faccium. Journal of Antimicrobial Chemotherapy 1997; 40:305-306. Linalool and cineole exhibit antibacterial activity against 17 and 16 out of 18 strains of gram positive and gram-negative bacteria tested, respectively. [see Pattnaik S, Subramanyam. Antibacterial and antifungal activity of aromatic constituents of essential oils. Microbios 1997; 89:39-46].

1. An organic and all natural method of treating, prevention, and relief of diaper dermatitis and other related skin irritations.

2. The composition according to claim 1 containing Beeswax wherein the proportion is 8-15% of the total formulation.

3. The composition according to claim 1 containing Meadowfoam Seed Oil wherein the proportion is 8%-10% of the total formulation.

4. The composition according to claim 1 containing Grapeseed Oil wherein the proportion is 60%-80% of the total formulation.

5. The composition according to claim 1 containing the herb Chamomile wherein the proportion is 0.01%-0.03% of the total formulation added as an extract or some form thereof.

6. The composition according to claim 1 containing the herb Calendula wherein the proportion is 0.01%-0.03% of the total formulation added as an extract or some form thereof.

7. The composition according to claim 1 containing the herb Comfrey wherein the proportion is 0.01%-0.03% of the total formulation added as an extract or some form thereof.

8. The composition according to claim 1 containing the herb Lavender wherein the proportion is 0.01%-0.03% of the total formulation added as an extract or some form thereof.

9. The composition according to claim 1 comprising of at least two vehicles or pharmaceutical carriers.

10. The composition according to claim 1 is in the form suitable for topical application.

11. The composition according to claim 1 is in the form but not limited to, cream, ointment, salve, lotion, solution, spray or bandage.
